(19) United States
(12) Patent Application Publication
(10) Pub. No.: US 2012/0173152 A1
(43) Pub. Date: Jul. 5, 2012

(54) GLUCOSE MANAGEMENT DEVICE AND METHOD OF USING THE SAME

(76) Inventors: Judd G. Butler, Sioux City, IA (US); Pamela J. Fleckenstein, Dakota Dunes, SD (US)

(21) Appl. No.: 13/073,026

(22) Filed: Mar. 28, 2011

Related U.S. Application Data
(60) Provisional application No. 61/427,883, filed on Dec. 29, 2010.

(51) Int. Cl. G01N 33/48; G06F 19/00

(52) U.S. Cl. 702/19

(57) ABSTRACT

A glucose management device and method of managing glucose using the same. The glucose management device has a housing with an input device and receives a vitro test element that determines a user's glucose level. Glucose measurement circuitry is electrically connected to the vitro test element to communicate data received from the vitro test element to a processor. The processor then prompts a user to input user information regarding units of insulin taken, other medication taken, and activity level into the input device so that the processor records the information for later downloading.

### CROSS REFERENCE TO RELATED APPLICATION

This application claims the benefit of U.S. Provisional Application No. 61/427,883 filed Dec. 29, 2010.

### BACKGROUND OF THE INVENTION

The invention is directed to glucose monitoring and more particularly a device that measures blood glucose levels, records units of insulin taken, other types of medication, whether intravenous, oral, inhaled and/or topical, and registers daily activity.

Devices for measuring blood glucose levels are known in the art. Typically, known devices determine a blood glucose level from a sample placed on a vitro test strip and registers the date, time, and blood sugar level. Based on this information, an individual may adjust their insulin dosage. Dosage information is logged by the individual on a separate sheet of paper or input into a separate computer.

While known test devices are helpful in determining glucose levels, they do not provide for the input of insulin dosage, they do not prompt the user to input units of insulin, other type of medication, or physical activity information, nor do they allow certain information that can be downloaded by third parties such as pharmacists and the like. Accordingly, a need exists in the art for a device and method that addresses these needs.

Thus, a principle object of the invention is to provide a glucose management device and method of using the same that improves data gathering.

### BRIEF SUMMARY OF THE INVENTION

A glucose management device that has a housing with an input device. The housing receives a vitro test element and also contains glucose measurement circuitry that is electrically connected to the vitro test element to communicate user information to a processor. After receiving the data provided by the vitro test element the processor will display the blood glucose level and then prompts by display and/or voice, a user to input units of insulin, other types of medication, and/or daily activity into the input device.

### BRIEF DESCRIPTION OF THE DRAWINGS

FIG. 1 is a schematic diagram of a glucose management device;

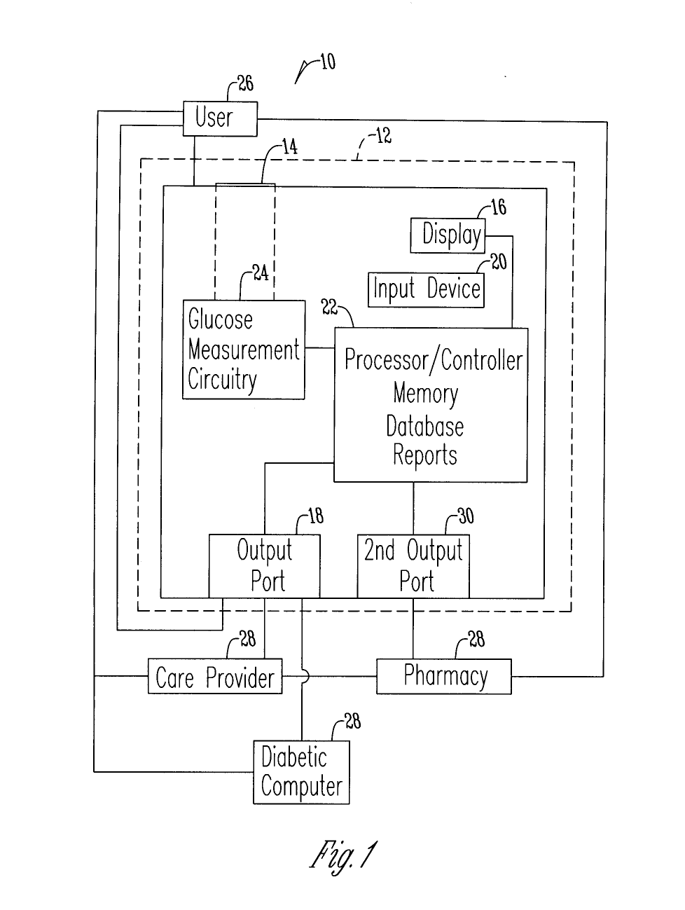

FIG. 2A is a partial decision flow diagram of a method of managing glucose steps; and

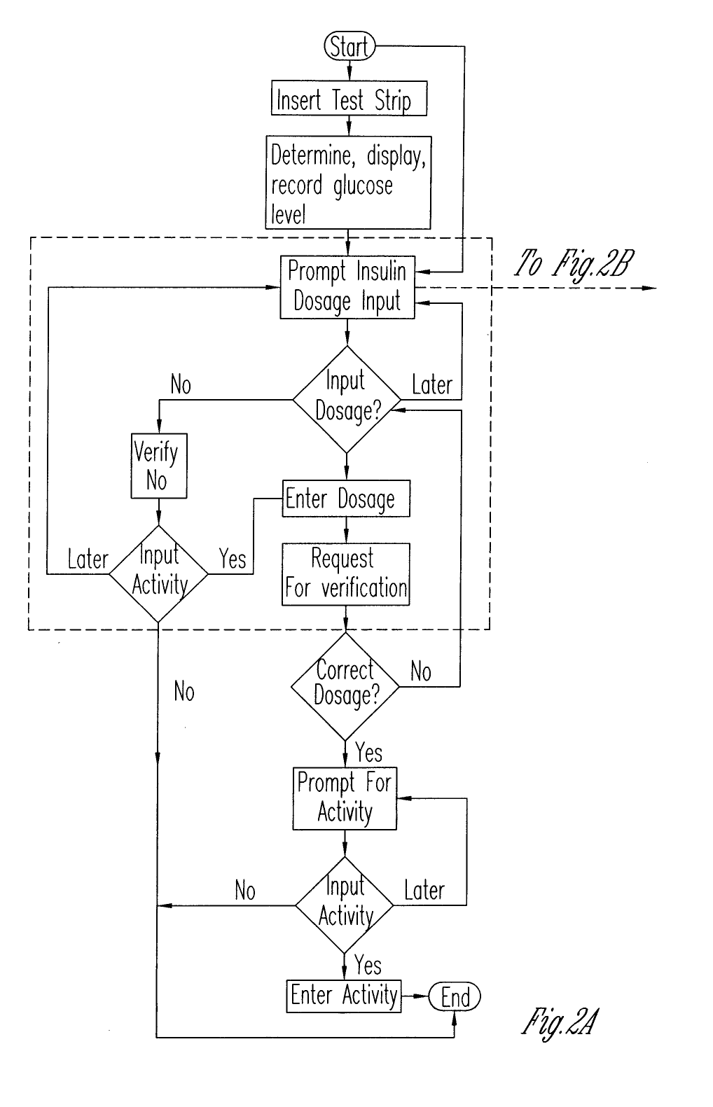

FIG. 2B is a partial decision flow diagram of a method of managing glucose steps.

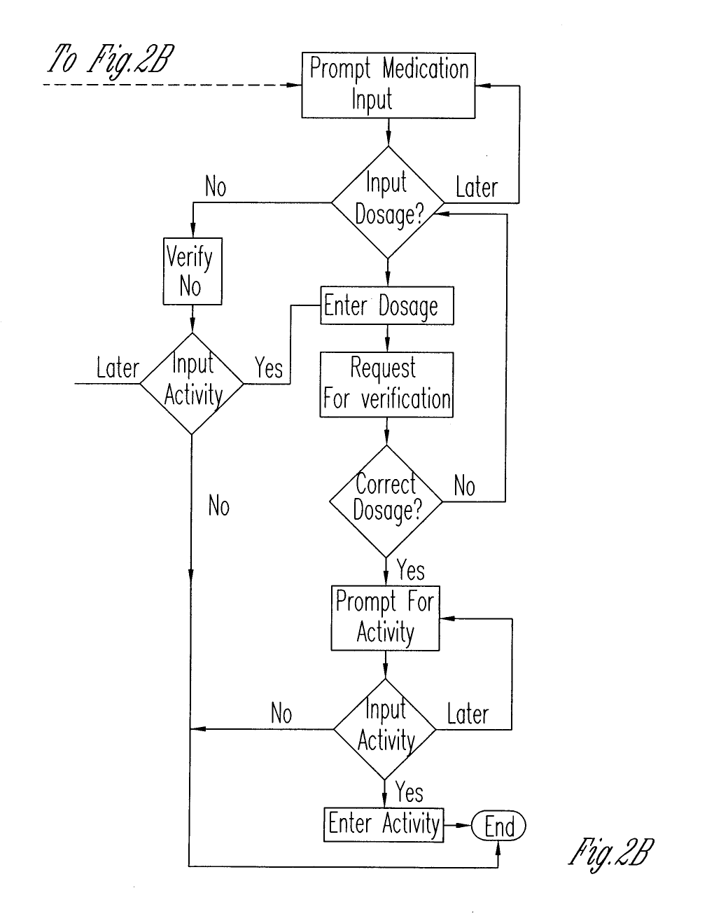

### DETAILED DESCRIPTION OF THE PREFERRED EMBODIMENTS

Referring to the figures, a glucose management device 10 has a housing 12 with a slot for insertion of a vitro test element or strip 14. On the exterior of the housing 12 is a display 16, an output port or interface 18, and an input device 20. Within the housing 12 is a processor/controller 22 having a memory with at least one database. Connected to the processor is glucose measurement circuitry 24 that interacts with the vitro test element 14 to provide data to the processor 22 to determine glucose level.

In operation, the vitro test element 14′such as a strip is inserted into the slot of the glucose management device 10 and a blood sample is placed on the end of the inserted strip 14. The glucose measurement circuitry 24 interacts with the sample on the test strip in the insertion slot and sends data to the processor 22. The processor 22 determines a glucose level based upon the data and displays and states the glucose level on the display 16 and stores the glucose level along with the date and time in a database within the memory. The processor 22 may also activate speech enabling software to audibly provide the glucose level for a user.

Next, the processor prompts a user 26 to input user information such as the number of units of insulin taken, other types of medication taken, and/or daily activity. This includes options regarding oral, topical, or mist/inhaled types of insulin or other types of medication. The processor 22 initiates the prompt by sending a signal to the display 16, a light, an alarm, a voice command speaker, or any combination of these or similar devices. In a preferred embodiment a user 26 has an option of declining the prompt, accepting the prompt, or requesting another prompt at a later time. If the user 26 declines the prompt the device will turn off. If another prompt is requested the processor 22 will repeat the prompting request at a later time until the user either declines or accepts the prompt. If the user 26 accepts the prompt, the user 26 will input the units of insulin, or other types of mediation into the input device 20. The input device 20 is of any type such as a keypad, voice recognition device, or the like.

Once the dosage is entered the information is received by the processor 22 and the processor 22 prompts the user 26 to verify the data that was input. If the information is incorrect the user re-enters the data. If the information is correct the processor 22 stores the information in the same or a different database and the meter is turned off.

The processor 22 also prompts the user 26 to input other types of medication, and/or an activity level. Similar to the units of insulin, the user has the option to decline, accept, or be prompted later. If accepted, the user 26 enters the data and the information is received by the processor 22 verified and stored in the same or a different database. This process is repeated over a protracted period of time creating a record for the user 26.

The record is maintained over time and can be downloaded in random through the output port/interface 18 to a diabetic user's computer, a care provider's computer or electronic medical record system 28. One of the advantages of this is that the care provider has access to a more complete history which enhances patient care, allows for a more in depth treatment plan, diet plan, and diabetic control by the user, care provider, diabetic trainer, and nutritionist.

Portions of the record also are downloaded to a pharmacist through the output/port interface. Medicare requires pharmacies to retrieve a log from users each time the strips are refilled. Because only a log of use of test strips is needed, and considering HIPPA regulations, the user could request a report be generated by the processor of only glucose measurements. Alternatively, this information could be saved in a separate database that is accessed through the output port 18 or a second output port 30 if needed. The report would be downloaded to the pharmacy's database and supplied to Medicare on request to show compliance with regulations and reduce or eliminate fraud.

Thus a glucose management device has been disclosed that at the very least meets all of the stated objectives.

What is claimed is:

1. A glucose management device comprising:
a housing have an input device and receiving a vitro test element;
glucose measurement circuitry electrically connected to the vitro test element;
a processor in communication with the glucose measurement circuitry to receive data provided by the vitro test element; and
wherein the processor prompts a user to input user information into the input device after the processor receives the data provided by the vitro test element.

2. The device of claim 1 wherein the processor prompts the user by sending a signal to a display on the housing.

3. The device of claim 1 wherein the user inputs insulin information.

4. The device of claim 3 wherein the insulin information is stored on a database of the processor.

5. The device of claim 4 wherein the insulin information is downloaded through an outlet port of the processor to a computer.

6. The device of claim 1 wherein the processor audibly states a blood glucose level.

7. The device of claim 2 wherein the display shows a blood glucose level.

8. The device of claim 1 wherein the user inputs information related to mediation and activity level.

9. A method of managing glucose steps comprising:
determining a glucose level of a user with a vitro test element;
communicating the glucose level of the user to a processor; and
prompting the user with the processor to input user information into an input device.

10. The method of claim 9 further comprising the step of inputting user information into the input device.

11. The method of claim 10 further comprising the step of recording the glucose level and user information in a database of the processor.

12. The method of claim 10 further comprising the step of prompting the user to input additional user information after the user information is inputted.

13. The method of claim 11 further comprising the step of downloading the user information from the database to a computer.

14. The method of claim 9 wherein the user information is insulin information.

15. The method of claim 9 wherein the user information is associated with medication and an activity level.
